(19) United States
(12) Patent Application Publication
(10) Pub. No.: US 2014/0227134 A1
(43) Pub. Date: Aug. 14, 2014

(54) ELECTRONICALLY CONTROLLED GAS MIXING UNIT FOR SUPPLYING A PURGE GAS TO AN OXYGENATOR

(71) Applicants: Thilo Joost, Friedberg (DE); Rainer Kobrich, Neulussheim (DE)

(72) Inventors: Thilo Joost, Friedberg (DE); Rainer Kobrich, Neulussheim (DE)

(73) Assignee: Maquet Vertrieb und Service Deutschland GmbH, Rastatt (DE)

(21) Appl. No.: 14/234,908

(22) PCT Filed: Jul. 27, 2012
(86) PCT No.: PCT/EP2012/064802

§ 371 Date: Apr. 14, 2014

(30) Foreign Application Priority Data
Jul. 27, 2011 (DE) 10 2011 052 189.5

(51) Int. Cl. A61M 1/16

(52) U.S. Cl. CPC A61M 1/1698; A61M 1/16
USPC 422/48; 210/188; 210/101; 210/85; 210/177

(57) ABSTRACT

The invention relates to an arrangement (10, 100) for removing carbon dioxide from an extracorporeal flow of blood and/or for oxygenating the extracorporeal flow of blood. The arrangement (10, 100) comprises an oxygenator (12), a first gas storage tank (24) in which a first gas is contained and at least a second gas storage tank (24) in which a second gas is contained. Further the device (10, 100) has a gas mixing unit for mixing a purge gas from the first and the second gas and an electronic control unit (30) for controlling the gas mixing unit (50). The control unit (30) comprises an input unit (40) by means of which the content of the first gas in the purge gas is adjustable by an operator, wherein the control unit (30) controls the gas mixing unit (50) such that the purge gas has the adjusted content of the first gas.

### CROSS REFERENCE TO RELATED APPLICATIONS

This application is entitled to the benefit of and incorporates by reference subject matter disclosed in International Patent Application No. PCT/EP2012/064802 filed on Jul. 27, 2012 and German Patent Application No. 10 2011 052 189.5 filed Jul. 27, 2011.

### TECHNICAL FIELD

The invention relates to an arrangement for removing carbon dioxide from an extracorporeal flow of blood and/or for oxygenating the extracorporeal flow of blood. The arrangement comprises a filter having a membrane, which separates a blood region from a gas region. The extracorporeal flow of blood is passed through the blood region and a gas flow of a purge gas is passed through the gas region. Further, the arrangement has a first gas storage tank in which a first gas is contained and at least a second gas storage tank in which a second gas, different from the first gas, is contained. Furthermore, the arrangement comprises a gas mixing unit for mixing the first and the second gas into a purge gas which is suppliable via a supply line to the gas region of the filter.

### BACKGROUND

In medicine, in the case of ill patients so-called oxygenators are used in order to remove carbon dioxide from the blood of these patients and, if indicated, to oxygenate the blood. Nowadays, oxygenators comprising a membrane by which a blood region is separated from a gas region are used almost exclusively. The blood is taken from a main vessel of the patients and is fed into the blood region of the oxygenator preferably by means of a blood pump or the blood pressure of the patient. At the same time, a purge gas is transported through the gas region, normally either pure oxygen or a mixture of oxygen and nitrogen being used as purge gas. In particular, a mixture of 21% oxygen and 79% nitrogen, so-called AIR, is used. Depending on the illness of the patient also further gases, differing from the previously mentioned gases, can be used as purge gas. The purge gas is in particular taken from wall supplies available in medical facilities and thus meets the requirements of medical gases.

As a result of the pressure gradient of the partial pressure or the concentration gradient of the carbon dioxide at the membrane, carbon dioxide is transported from the blood region through the membrane into the gas region, whereas as a result of the pressure gradient of the partial pressure of the oxygen or the corresponding concentration gradient of the oxygen, the oxygen is transported through the membranes from the gas region into the blood region so that the blood is oxygenated and at the same time carbon dioxide is removed from the blood. The amount of carbon dioxide that is removed from the blood per unit of time, and/or the amount of oxygen with which the blood is oxygenated per unit of time depend i.a. on the flow rate of the purge gas through the gas region, on the flow rate of the blood through the blood region as well as on the composition of the purge gas.

In known arrangements, normally two gas storage tanks are provided in which different gases are contained. Via simple mechanical gas mixing units, so-called gas blenders, the purge gas can be mixed together from these gases. For this purpose, in particular adjustment wheels are provided, which can be operated manually, wherein via the adjustment wheels the opening of a valve of the gas storage tanks is adjusted and thus the composition is changed by the amount of gas removed from the gas storage tank.

A problem of such known gas blenders is that by means of them the composition of the purge gas and/or the flow rate of the purge gas, with which it flows through the gas region of the oxygenator, can only be adjusted very inaccurately. Further this manual adjusting is complex and error-prone.

### SUMMARY

It is the object of the invention to provide an arrangement for removing carbon dioxide from an extracorporeal flow of blood and/or for oxygenating the extracorporeal flow of blood, in which the composition of a purge gas is exactly adjustable in an easy manner.

This object is solved by an arrangement with the feature of claim 1. Advantageous developments of the invention are specified in the dependent claims.

According to the invention the arrangement comprises an electronic control unit for controlling the gas mixing unit, which comprises an input unit by means of which the content of the first gas in the purge gas is adjustable by an operator.

The control unit controls the gas mixing unit such that the purge gas has the adjusted content of the first gas. By such an electronically controlled gas mixing unit on the one hand an easy comfortable handling is achieved and on the other hand a precise adjustability of the composition of the purge gas is guaranteed. In particular, the operator does not have to calculate himself/herself anymore, how much which valve of which gas storage tank has to be opened to obtain the desired flow rate of the purge gas and the desired composition of the purge gas.

The filter comprises in particular an oxygenator via which the removal of the carbon dioxide or the supply of the oxygen is possible in an easy manner. The flow of blood is in particular passed through the blood region of the filter by means of a blood pump. Further, also a gas transport unit for generating the gas flow through the gas region can be provided. Alternatively, the gas flow of the purge gas can also be generated only by the pressure with which the gases are contained in the gas storage tanks.

The gas mixing unit comprises in particular a first proportional valve for adjusting the flow of volume of the first gas removed from the first gas storage tank and a second proportional valve for adjusting the flow of volume of the second gas removed from the second gas storage tank. The control unit controls the first proportional valve and/or the second proportional valve such that the purge gas comprises the desired content of the first gas and thus also the desired content of the second gas.

The control unit determines in particular as a function of the adjusted content of the first gas a first set point for the first proportional valve and/or a second set point for the second proportional valve and adjusts this first set point at the first proportional valve and/or the second set point at the second proportional valve. Thus, the set points do not have to be elaborately determined or tested by the operator, but a fully automatic adjustment of the desired composition of the purge gas by the electronic control unit is possible.

In a particularly advantageous embodiment of the invention, a third gas storage tank in which a third gas is received and at least a fourth gas storage tank in which a fourth gas is received are provided. Hereby it is achieved that the number of the different available gases is increased, so that the used purge gas can be adapted to the state of the patient particularly precisely, without the gas storage tanks having to be exchanged for this purpose. The gas mixing unit mixes the purge gas to this end from the first gas, the second gas, the third gas and/or the fourth gas, wherein via the input unit the content of at least three of the four gases is adjustable and the control unit controls the gas mixing unit such that the purge gas has the adjusted content of the gases.

It is particularly advantageous, if the gas mixing unit comprises a third proportional valve for adjusting the flow of volume removed from the third gas storage tank and/or a fourth proportional valve for adjusting the flow of volume of the fourth gas removed from the fourth gas storage tank. For adjusting the content of the third gas adjusted via the input unit the control unit controls the third proportional valve or the fourth proportional valve correspondingly. In particular, the control unit determines again as a function of the adjusted content of the third gas a third set point for the third proportional valve and/or as a function of the adjusted content of the fourth gas a fourth set point for the fourth proportional valve and adjusts the corresponding set point at the third or the fourth proportional valve.

Via the input unit in particular the content of all four gases is respectively adjustable, so that the operator irrespective of which gas content he/she wants to adjust, can insert it directly and does not have to calculate another content first in order to indirectly adjust the desired content of the desired gas in this manner.

As first, second, third or fourth gas in particular oxygen, carbon dioxide, nitrogen, neon, xenon, helium, krypton or argon can be contained in the respective gas storage tanks. Alternatively, also mixtures of at least two of the previously mentioned gases can be stored in the gas storage tanks, for example in one of the gas storage tanks a mixture of 21% oxygen and 79% nitrogen, so-called AIR, can be contained. Likewise, in one of the gas storage tanks a mixture of 21% oxygen and 79% helium, so-called heliox, can be contained.

The input unit comprises in particular a touchscreen, so that the control unit can be controlled in an easy manner. Further, such a touchscreen makes it possible that via a single unit both data can be input, in particular data via which the compositions of the purge gas can be input, as well as information can be output to the operator.

Via the input unit in particular a desired value for the flow of volume of the purge gas is adjustable, with which the purge flows through the gas region of the filter. The control unit controls the first proportional valve, the second proportional valve, the third proportional valve and/or the proportional valve such that the gas flow of the purge gas mixed together from the different gases contained in the gas storage tanks has the adjusted flow of volume. The gas mixing unit is in particular designed such that by means thereof flows of volume between 0.1 l/min and 20 l/min are adjustable.

Downstream of the gas mixing unit and upstream of the filter in particular a sensor unit is provided for determining an actual value of the content of the first gas in the purge gas, an actual value of the content of the second gas in the purge gas, an actual value of the content of the third gas in the purge gas and/or an actual value of the content of the fourth gas in the purge gas. Thus, it can be monitored in an easy manner, if the composition of the purge gas from the individual gases actually corresponds to the desired composition. Additionally or alternatively, via the sensor unit also an actual value of the flow of volume of the purge gas can be determinable, so that said actual value can also be monitored in an easy manner.

The control unit compares the actual value of the content of one of the gases preferably respectively with the correspondingly adjusted desired value of this gas and controls the gas mixing unit as a function of this result of this comparison. In particular, the control unit controls the gas mixing unit via a closed control loop such that the purge gas actually comprises the adjusted contents of the individual gases. Likewise, the control unit preferably compares the determined actual value of the flow of volume with the adjusted desired value of the flow of volume and controls the gas mixing unit as a function of the result of this comparison, in particular again via a closed control loop.

Additionally or alternatively, the input unit can output a piece of information about at least one, preferably all, actual values determined by means of the sensor unit to the operator, so that he/she can monitor in an easy manner, if the composition and/or the flow of volume corresponds to the adjusted presettings. In particular, via the touchscreen a graphic output of the individual values are illustrated, for example as bar diagrams.

Downstream of the filter a further sensor unit can be arranged for determining a further actual value of the content of the first gas in the purge gas, a further actual value of the content of the second gas in the purge gas, a further actual value of the content of the third gas in the purge gas, a further actual value of the content of the fourth gas in the purge gas and/or a further actual value of the flow of volume of the purge gas. In this manner, it can in particular be determined, if from the flow of blood a part of at least one of the gases has been received and if this is the case, which amount.

Via the input unit in particular a piece of information about the further actual values determined by means of the further sensor unit are output to the operator. In particular, respectively a difference value is determined via the actual value determined by means of the one sensor unit arranged upstream of the filter and the corresponding further actual value determined by means of the further sensor unit arranged downstream of the filter and this difference value is shown to the operator, so that the change of the composition of the purge gas and/or the flow rate of the purge gas through the flowing through of the gas region of the filter is obvious in an easy manner.

Additionally or alternatively, the control unit can compare one or several of the actual values determined by means of the further sensor unit with the corresponding preset values or control the gas mixing unit as function of the result of this comparison.

The first gas, the second gas, the third gas and/or the fourth gas comprise in particular a medicament, so that via the filter also a medication of the patient is possible.

Downstream of the gas mixing unit and upstream of the filter preferably a vaporization unit for vaporizing a volatile medicament is provided by means of which the volatile medicament can be vaporized, wherein the vaporized medicament is supplied to the purge gas and is thus passed together with the gases removed from the gas storage tanks through the gas region of the filter, so that the vaporized medicament can get into the flow of blood of the patient via the filter.

Downstream of the gas mixing unit and upstream of the filter in particular a heating element for heating the purge gas and/or a humidifying unit for humidifying the purge gas is arranged. By heating the purge gas it is achieved that the temperature difference between the purge gas and the flow of blood is balanced and at least minimized, so that no cooling of the patient occurs. The humidifying unit causes that the relatively dry purge gas has a higher saturation of vapour, in particular a saturation of vapour of 100%, so that on the one hand a removal of water from the flow of blood and on the other hand a deposition of water at the membrane are prevented.

Upstream of the filter preferably a temperature sensor for determining the actual value of the temperature of the purge gas and/or a humidifying sensor for determining the air humidity of the purge gas is arranged, so that the temperature or the humidity of the purge gas can be monitored. In particular, the control unit compares the actual values of the temperature and the air humidity determined by this sensor with preset values and controls the heating unit or the humidifying unit such that they heat or humidify the purge gas such that it has the preset temperature or the preset air humidity.

Additionally or alternatively, also downstream of the filter a further temperature sensor for determining the temperature of the purge gas and/or a further humidifying sensor for determining the air humidity of the purge gas can be provided. The actual values of the temperature and/or air humidity which were determined both via the sensors arranged upstream of the filter and arranged downstream of the filter, are preferably again shown via the touchscreen of the control unit to the operator, so that the operator cannot only monitor the composition and the flow rate of the purge gas, but also the temperature and air humidity before and after the passage of the filter.

Further it is advantageous, if a return line for returning the purge gas from the filter into the supply line is provided, so that the purge gas can be used several times. Thus, not always new purge gas has to be supplied, whereby costs are saved. Alternatively, the purge gas can also be recycled and/or disposed after the passage of the filter.

### BRIEF DESCRIPTION OF THE DRAWINGS

Further features and advantages of the invention result from the following description which explains the invention on the basis of embodiments in connection with the attached Figures, in which:

FIG. 1 shows a schematic illustration of an arrangement for removing carbon dioxide from an extracorporeal flow of blood and/or for oxygenating the extracorporeal flow of blood according to a first embodiment; and

FIG. 2 shows a schematic illustration of an arrangement for removing carbon dioxide from an extracorporeal flow of blood and/or for oxygenating the extracorporeal flow of blood according to a second embodiment.

### DETAILED DESCRIPTION

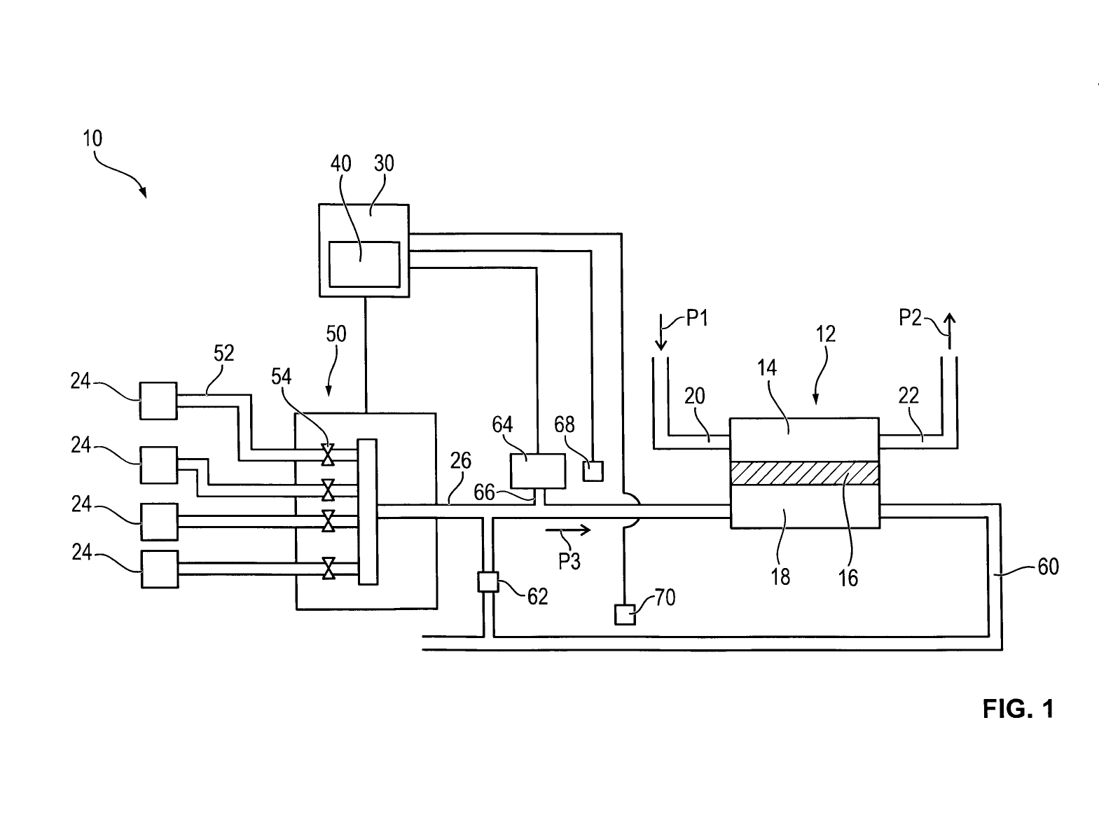

In FIG. 1, a schematic illustration of an arrangement 10 for removing carbon dioxide from an extracorporeal flow of blood of a patient and for oxygenating the extracorporeal flow of blood is illustrated. The arrangement 10 comprises a filter designed as oxygenator 12, which has a blood region 14 and a gas region 18 separated from this blood region 14 by a membrane 16. The extracorporeal flow of blood is passed through the blood region 14 according to the arrows P1 and P2, for which a supply line 20 and a discharge line 22 are provided. A blood pump can also be provided by means of which the flow of blood through the blood region 14 is generated.

A purge gas is passed through the gas region 18, which is indicated by the arrow P3. Further, the arrangement 20 comprises a gas mixing unit 50 electronically controlled via a control unit 30, which is also referred to as gas mixing bank. The arrangement 10 has four gas storage tanks 24 which are respectively connected via a line 52 with the gas mixing unit 50. In the gas storage tanks 24 respectively a gas or a mixture of several gases is contained.

The gas mixing unit 50 has four proportional valves, one of which is exemplarily referred to with the reference sign 54. Respectively one of the proportional valves 54 serves for regulating the flow of volume which is removed from one of the gas storage tanks 24. The gases removed from the individual gas storage tanks 24 via the proportional valves 54 are mixed together into the purge gas and are supplied via a supply line 26 to the gas region 18 of the oxygenator 12.

Due to a partial pressure gradient or a concentration difference existing between individual components of the purge gas and the corresponding component in the flow of blood this component is passed through the membrane 16 from the gas region 18 into the blood region 14 or vice versa. In particular, in this manner oxygen is passed from the gas region 18 into the blood region 14, so that the flow of blood is oxygenated. Vice versa, carbon dioxide is passed from the blood region 14 into the gas region 18 through the membrane 16, so that carbon dioxide is removed from the flow of blood. The transfer capacity of the oxygen or the carbon dioxide, i.e. the amount of carbon dioxide or oxygen which is supplied or removed from the flow of blood per unit of time depends i.a. on the flow rate of the flow of blood, the flow of volume of the purge gas and the composition of the purge gas.

In particular, it can also be controlled via the composition of the purge gas, if only oxygen is supplied, only carbon dioxide is removed or if both is done simultaneously. Therefore, it is important that the composition of the purge gas and the flow of volume of the purge case can be adjusted as precisely as possible, which is easily possible by means of the previously described electronically controlled gas mixing unit 50.

In the gas storage tanks 24 as gases in particular nitrogen, carbon dioxide, oxygen, helium, xenon, neon, argon, krypton, a mixture of 21% oxygen and 79% nitrogen, a mixture of 21% oxygen and 79% helium or further medical gases or gas mixtures are contained. Via the proportional valves 54 the content of the gases in the purge gas removed from the gas storage tanks 24 and thus also the flow of volume of the purge gas can be adjusted automatically and precisely. The control unit 30 in particular has a touchscreen 40 via which an operator of the arrangement 10 can adjust the desired composition of the purge gas, i.e. the contents of the individual gases, in an easy manner. Further, the operator can adjust the flow of volume of the purge gas via the control unit 30. The control unit 30 determines in particular variables for the individual proportional valves 54 as a function of the values adjusted by the operator and controls the gas mixing unit 50 correspondingly. Thus, the operator can insert the contents in an easy manner directly and does not elaborately have to calculate or find out by trying the variables of the individual proportional valves 54, as it is for example the case with mechanical gas blenders.

In an alternative embodiment of the invention, also more than four gas storage tanks 24, for example five gas storage tanks 24, or less than four gas storage tanks 24, for example two gas storage tanks 24, can be provided.

After the passage of the gas region 18 of the oxygenator 12 the purge gas is passed back again via a return line 60 to the supply line 26, so that the purge gas can be used several times for flowing through the gas region 18 and thus not always new gas has to be removed from the gas storage tank 24. In particular, the returned gas is mixed with gas newly removed from the gas storage tanks 24 before it is again supplied to the oxygenator 12. The return line 60 is preferably connected to the gas mixing unit 50, so that the returned purge gas can be mixed via the gas mixing unit 50 with gases possibly removed from the storage tanks 24. Alternatively, the connection of the return line 60 with the supply line 26 can also be made downstream of the gas mixing unit 50.

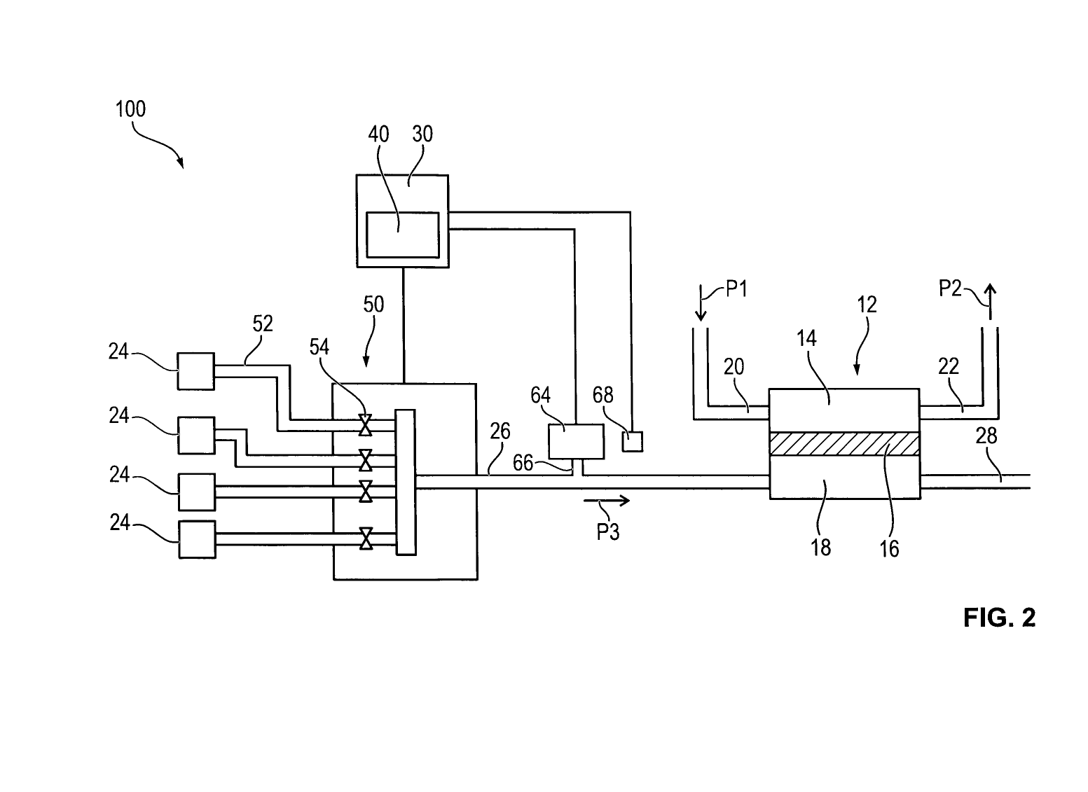

In a second embodiment, which is shown in FIG. 2, instead of a return line 60 only a discharge line 28 of the arrangement 100 is provided, via which the purge gas after flowing through the gas region 18 is supplied to a recycling or disposal unit.

In the region of the return line 60 in particular a ventilator 62 is arranged by means of which the flow of the purge gas is maintained. In an alternative embodiment, the gas flow of the purge gas can also be generated only by the pressure with which the gases are contained in the gas storage tanks 24.

Furthermore, a vaporization unit 64 is provided by means of which volatile medicaments can be vaporized. The vaporized medicament is supplied via a connection line 66 to the supply line 26, so that also the vaporized medicament can be transferred via the oxygenator 12 to the flow of blood and thus be administered to the patient. The vaporization unit 64 is in particular also controlled via the control unit 30.

Further, the arrangement 10 comprises two sensor units 68, 70, wherein one of the sensor units 68 is arranged upstream of the oxygenator 12 in the region of the supply line 26 and the other sensor unit 70 is arranged downstream of the oxygenator 12 in the region of the return line 60 or the discharge line 28. By means of the sensor units 68, 70 in particular the composition of the purge gas can be determined, i.e. it is determined, how high the actual value of the contents of the individual gases in the purge gas are. The determined actual values are in particular compared with the desired values preset by the operator of the control unit 30, wherein the control unit 30 controls the gas mixing unit 50 as a function of the result of this comparison in the form of a closed control loop such that the actual content of the respective gas corresponds to the preset desired value.

Additionally, via the sensor units 68, 70 also respectively the flow rate of the purge gas can be determined, wherein the flow unit preferably again compares the determined actual value of the flow rate with the preset desired value and controls the individual proportional valves 54 such that the actual flow rate corresponds to the desired value.

Additionally or alternatively, the actual values determined via the sensor units 68, 70 can also be shown to the operator via the touch screen 40, so that he/she can easily monitor the proper functioning of the arrangement 10. In particular, a difference value from the actual values of the respective contents of the gases determined upstream and downstream of the oxygenator 12 are indicated, preferably graphically, to the operator, so that he/she can monitor in an easy manner how much carbon dioxide has been removed from the flow of blood or how much oxygen is supplied to the flow of blood.

Furthermore, the sensor units 68, 70 can also comprise temperature sensors for determining the temperature of the purge gas and/or humidifying sensors for determining the air humidity of the purge gas. Likewise, a heating unit for heating the purge gas and/or a humidifying unit for increasing the air humidity of the purge gas can be provided upstream of the oxygenator.

Although various embodiments of the present invention have been described and shown, the invention is not restricted thereto, but may also be embodied in other ways within the scope of the subject-matter defined in the following claims.

What is claimed is:

1. An arrangement for removing carbon dioxide from an extracorporeal flow of blood and/or for oxygenating the extracorporeal flow of blood,
with a filter comprising a membrane that separates a blood region through which the extracorporeal flow of blood is passed from a gas region through which a gas flow of a purge gas is passed,
a first gas storage tank in which a first gas is contained and at least a second gas storage tank in which a second gas, different from the first gas, is contained,
a gas mixing unit for mixing the first gas and the second gas into the purge gas, and
with a supply line for supplying the purge gas to the gas region of the filter,
wherein an electronic control unit for controlling the gas mixing unit is provided,
the control unit comprises an input unit by means of which the content of the first gas in the purge gas is adjustable by an operator, and
that the control unit controls the gas mixing unit such that the purge gas has the adjusted content of the first gas.

2. The arrangement according to claim 1, wherein the gas mixing unit comprises a first proportional valve for adjusting the flow of volume of the first gas removed from the first gas storage tank and a second proportional valve for adjusting the flow of volume of the second gas removed from the second gas storage tank, and that the control unit controls the first proportional valve and/or the second proportional valve for adjusting the content of the first gas adjusted via the input unit.

3. The arrangement according to claim 2, wherein the control unit determines as a function of the adjusted content of the first gas a first set point for the first proportional valve and/or a second set point for the second proportional valve, and that the control unit adjusts the first set point at the first proportional valve and/or adjusts the second set point at the second proportional valve.

4. The arrangement according to claim 1, wherein a third gas storage tank in which a third gas is contained and at least a fourth gas storage tank in which a fourth gas is contained are provided, that the gas mixing unit mixes the purge gas from the first gas, the second gas, the third gas and/or the fourth, that the content of at least three gases is adjustable via the input unit, and that the control unit controls the gas mixing unit such that the purge gas has the adjusted contents of the gases.

5. The arrangement according to claim 4, wherein the gas mixing unit comprises a third proportional valve for adjusting the flow of volume of the third gas removed from the third gas storage tank and/or a fourth proportional valve for adjusting the flow of volume of the fourth gas removed from the fourth gas storage tank, and that the control unit controls the third proportional valve and/or the fourth proportional valve for adjusting the adjusted content of the gas.

6. The arrangement according to claim 5, wherein the control unit determines a third set point for the third proportional valve as a function of the adjusted content of the third gas and/or a fourth set point for the fourth proportional valve as a function of the adjusted content of the fourth gas, and that the control unit adjusts the third set point at the third proportional valve and/or adjusts the fourth set point at the fourth proportional valve.

7. The arrangement according to claim 4, wherein the content of all four gases is adjustable via the input unit.

8. The arrangement according to claim 1, wherein the first gas, the second gas, the third gas or the fourth gas is oxygen, carbon dioxide, nitrogen, at least one noble gas or a mixture of at least two of these gases.

9. The arrangement according to claim 1, wherein the input unit comprises a touchscreen.

10. The arrangement according to claim 1, wherein via the input unit a flow of volume of the purge gas, in particular in the range between 0.1 l/min and 20 l/min, is adjustable, and that the control unit controls the first proportional valve, the second proportional valve, the third proportional valve and/or the fourth proportional valve such that the purge gas has the adjusted flow of volume.

11. The arrangement according to claim 1, wherein downstream of the gas mixing unit and upstream of the filter a sensor unit is arranged for determining an actual value of the content of the first gas in the purge gas, an actual value of the content of the second gas in the purge gas, an actual value of the content of the third gas in the purge gas, an actual value of the content of the fourth gas in the purge gas and/or an actual value of the flow of volume of the purge gas.

12. The arrangement according to claim 11, wherein the control unit compares the actual value of the content of the first gas, the actual value of the content of the second gas, the actual value of the content of the third gas, the actual value of the content of the fourth gas and/or the actual value of the flow of volume of the purge gas with the respectively corresponding value adjusted via the input unit, and that the control unit controls the gas mixing unit as a function of the result of this comparison, in particular in the form of a closed control loop.

13. The arrangement according to claim 11, wherein the input unit outputs a piece of information about the actual values determined by means of the sensor unit to the operator.

14. The arrangement according to claim 1, wherein downstream of the filter a further sensor unit is arranged for determining a further actual value of the content of the first gas in the purge gas, a further actual value of the content of the second gas in the purge gas, a further actual value of the content of the third gas in the purge gas, a further actual value of the content of the fourth gas in the purge gas and/or a further actual value of the flow of volume of the purge gas, and that the input unit outputs a piece of information about the further actual values determined by means of the further sensor unit to the operator and/or the control unit compares the further actual value of the content of the first gas, the further actual value of the content of the second gas, the further actual value of the content of the third gas, the further actual value of the content of the fourth gas and/or the further actual value of the flow of volume of the purge gas with the respectively corresponding adjusted value and controls the gas mixing unit as a function of the result of this comparison.

15. The arrangement according to claim 14, wherein the control unit determines respectively a difference value between said one actual value and said corresponding further actual value and outputs information about the determined difference values, in particular in form of a graphic, via the input unit to the operator.

16. The arrangement according to claim 1, wherein the first gas, the second gas, the third gas and/or the fourth gas respectively comprise a medicament.

17. The arrangement according to claim 1, wherein downstream of the gas mixing unit and upstream of the filter a vaporization unit for vaporizing a volatile medicament is provided and that the vaporized medicament is suppliable to the purge gas by means of a supply unit.

18. The arrangement according to claim 1, wherein downstream of the gas mixing unit and upstream of the filter a heating unit for heating the purge gas and/or a humidifying unit for humidifying the purge gas are arranged.

19. The arrangement according to claim 1, wherein upstream of the filter a temperature sensor for determining an actual value of the temperature of the purge gas and/or a humidifying sensor for determining the air humidity of the purge gas are arranged.

20. The arrangement according to claim 1, wherein downstream of the filter a further temperature sensor for determining the temperature of the purge gas and/or a further humidifying sensor for determining the air humidity of the purge gas are provided.

21. The arrangement according to claim 1, wherein a return line for returning the purge gas from the filter into the supply line is provided.
